(19) United States
(12) Patent Application Publication
(10) Pub. No.: US 2002/0151751 A1
(43) Pub. Date: Oct. 17, 2002

(54) Process for the preparation of substituted amines by hydrogenation of substituted organic nitro compounds

(76) Inventors: Daniel Ostgard, Kleinostheim (DE); Monika Berweiler, Maintal (DE); Stefan Roeder, Sinntal (DE)

Correspondence Address: SMITH, GAMBRELL & RUSSELL, LLP, 1850 M STREET, N.W., SUITE 800, WASHINGTON, DC, 20036, US

(21) Appl. No.: 10/046,537

(22) Filed: Jan. 16, 2002

(30) Foreign Application Priority Data
Jan. 16, 2001 (DE) 101 01 647.6

(51) Int. Cl. C 07C 2 9/36

(52) U.S. Cl. 564/420; 564/422

(57) ABSTRACT

A process for the preparation of substituted amines by catalytic hydrogenation of substituted organic nitro compounds with hydrogen or hydrogen-containing gas mixtures in the presence of a shaped Raney catalyst as the hydrogenation catalyst, wherein the Raney catalyst is in the form of hollow bodies or shell-activated tablets. Nickel, cobalt, copper, iron, platinum, palladium or ruthenium are preferably used as catalytically active constituents.

The invention relates to a process for the preparation of substituted amines from substituted organic nitro compounds, comprising catalytic hydrogenation of substituted organic nitro compounds with hydrogen or hydrogen-containing gases in the presence of a shaped hydrogenation catalyst of the Raney type.

According to the invention, substituted amines are understood as meaning compounds which contain further substituents in addition to exactly one amino group. These substituents can be, for example, alkyl groups or halogen substituents, and also further amino groups. The compounds on which they are based, that is to say the unsubstituted compounds, are n-alkanes and/or unsubstituted mono- or polynuclear aromatics.

Amines are an important substance class in organic chemistry. They are used, for example, as starting materials for the preparation of solvents, surfactants, bactericides, corrosion protection agents, antifoams, additives, pharmaceuticals or dyestuffs. They are furthermore of great importance for the preparation of polyamide and polyurethane plastics.

Raney catalysts are often preferred in the preparation of amines by hydrogenation of organic nitro compounds because of their good catalytic properties. Raney catalysts, which are also called activated metal catalysts, comprise an alloy of at least one catalytically active metal and at least one metal which can be leached out with alkalis. Aluminium is predominantly employed for the alloy component which is soluble in alkalis, but other metals, such as, for example, zinc and silicon, can also be used. The component which can be leached out is dissolved out by addition of alkalis to the alloy, as a result of which the catalyst is activated.

Numerous inventions for the preparation of amines by catalytic hydrogenation of organic nitro compounds with the aid of Raney catalysts are known. Various Raney catalysts, or more precisely catalysts with various active metals or metal combinations, are employed here, depending on the process.

For example, the document EP 0 789 018 describes a process for the hydrogenation of halogen-substituted nitroaromatics with the aid of an iron-containing Raney nickel catalyst. It becomes clear from the examples that the process described is preferably carried out in the batch process with the aid of powder catalysts.

The document U.S. Pat. No. 5,811,584 describes the preparation of an aliphatic compounds, more precisely an aliphatic diamine disubstituted by methyl. In this case, a Raney catalyst with cobalt as the catalytically active component is preferably used. In this process also, the hydrogenation is carried out with the aid of Raney powder catalysts.

Raney powder catalysts have the disadvantage that they can be employed only in the batch process or at best in the semi-continuous process if sufficient conversion rates are to be achieved under moderate reaction conditions. Furthermore, the catalyst must be separated off from the reaction media in an expensive manner after the catalytic reaction. For these reasons also, it is preferable to carry out the preparation of amines by hydrogenation of organic nitro compounds with the aid of shaped Raney catalysts and where possible in a continuous process. Fixed bed catalysts which, in addition to a good catalytic activity, must also have a sufficiently good strength for the continuous operation are needed for this purpose.

This disadvantage is avoided in the hydrogenation of nitro compounds such as is described in the document EP 0 842 699. For the hydrogenation, a shaped Raney catalyst with a metal of sub-group 8 as the catalytically active component and comprising a high content of macropores is used. The high content of macropores contributes towards reducing the bulk density and therefore towards a high catalyst activity compared with other Raney catalysts. A disadvantage of the method described in the document EP 0842699, however, is that the hydrogenation catalyst used still has a relatively high bulk density of above approx. 1.3 g/ml. This results in a relatively low activity of the catalyst with respect to the weight of the catalytically active metal employed.

The document DE 199 33 450.1 describes metal catalysts which are in the form of hollow bodies, preferably in the form of hollow spheres. These catalysts have a low bulk density of 0.3 to 1.3 g/ml. In addition to the catalysts, their use in hydrogenation reactions is furthermore described. The examples describe an activity test for the hydrogenation of nitrobenzene to aniline, in which the hydrogen consumption and therefore the activity of the catalyst per gram of catalyst is significantly higher if catalysts in the form of hollow spheres are used than if a comparison catalyst is used. However, the use of the catalysts described for the preparation of substituted amines by hydrogenation of substituted nitro compounds is not mentioned. This is of great important inasmuch as side reactions can often occur in the hydrogenation of substituted nitro compounds to substituted amines. Thus, for example, in the hydrogenation of nitroaromatics substituted by halogen or substituted by sulfane groups, dehalogenations or splitting off of sulfane groups are often observed, so that contents of undesirable unsubstituted aromatic amines which are too high are obtained due to a low selectivity of the catalyst. It cannot thus be necessarily assumed that by hydrogenation of substituted nitro compounds correspondingly substituted amines are to be achieved in good to very good yields.

The object of the invention is therefore to develop a process for the preparation of substituted amines by catalytic hydrogenation of substituted nitro compounds, in which the hydrogenation is carried out with a shaped hydrogenation catalyst of the Raney type which, with an adequate strength of the catalytically active layer and a substantially lower bulk density than comparable catalysts, has the same or a better hydrogenating activity than the catalysts used hitherto. Another aim of the invention is to achieve the same or better conversion rates of the starting materials using less catalyst material compared with known processes.

The invention has shown that the preparation of substituted amines by hydrogenation of substituted organic nitro compounds is possible with significantly higher conversion rates per unit weight of catalyst with the aid of the Raney catalysts in the form of hollow bodies described in the document DE 199 33 450.1 than with comparable catalysts. This observation is surprising in that it cannot necessarily be assumed that the Raney catalysts in the form of hollow bodies have the required activities and selectivities in the specific case of hydrogenation of substituted organic nitro compounds.

The invention provides a process for the preparation of substituted amines by catalytic hydrogenation of substituted organic nitro compounds with hydrogen or hydrogen-containing gas mixtures in the presence of a shaped Raney catalyst as the hydrogenation catalyst, which is characterized in that the Raney catalyst is in the form of hollow bodies. The process according to the invention has the advantage that substituted amines can be prepared with equally good or higher yields using significantly smaller amounts of catalyst than has hitherto been possible according to the prior art.

One of the advantages on which the invention is based is achieved by the use of Raney catalysts in the form of hollow bodies. The preparation of the catalysts used in the process according to the invention can be carried out according to the method described in DE 199 33 450.1. According to this method, a mixture of an alloy powder of a catalytically active metal with a metal which can be leached out, preferably aluminium, an organic binder and optionally an inorganic binder, water and promoters is applied to spheres which are made of a material which can preferably be removed by means of heat. Polystyrene foam spheres can preferably be used. The mixture comprising the metal alloy can preferably be applied to the polymer spheres in a fluidized bed. 0-10 wt. % polyvinyl alcohol and/or 0-3 wt. % glycerol can preferably be employed as the organic binder. The coated polymer foam spheres are then calcined above 300° C., preferably in a range from 450 to 1300° C., in order to remove the polymer foam by means of heat and to sinter the metal. The hollow spheres acquire a stable form as a result. After the calcining, the catalysts in the form of hollow spheres are activated by treatment with basic solutions, preferably alkali metal or alkaline earth metal hydroxides in water, in particular aqueous sodium hydroxide solution. The catalysts obtained in this way have bulk densities of between 0.3 and 1.3 kg/l.

For the process according to the invention, it is preferable for the Raney catalysts in the form of hollow bodies to comprise nickel, cobalt, copper, iron, platinum, palladium, ruthenium and/or mixtures of these metals as catalytically active constituents. Other variants of the preferred catalyst are shell-activated tablets of Raney alloys according to the instructions in EP 0 648 534 A1. The alloys used can be cooled either slowly or rapidly.

Those Raney catalysts which have been activated by leaching out aluminium, silicon and/or zinc, in particular aluminium, by means of alkalis are preferably used in the preparation according to the invention of substituted amines. The activation can preferably be carried out with aqueous solutions of sodium hydroxide. In this case, the weight ratio of water to alkali metal hydroxide is in general approximately 10:1 to about 30:1, preferably approximately 15:1 to 25:1. The molar ratio of alkali metal hydroxide to aluminium is as a rule 1:1 to approximately 6:1, preferably approximately 1.5:1 to approximately 4:1.

According to the invention, the process is carried out with catalysts in the form of hollow bodies, extrudates, granules, fibrous tablets or shell-activated tablets. It is preferable that the Raney catalysts is in the form of hollow spheres. Hollow spheres, extrudates, granules, fibrous tablets or shell-activated tablets are usually easy to produce and have a high breaking strength. Extrudates, granules, fibrous tablets or shell-activated tablets can preferably be employed for exothermic reactions because of their good heat exchange properties.

An important advantage of the process according to the invention is that the Raney catalysts used have the more optimized bulk density for the corresponding reaction, as with the Raney catalysts known from the prior art for the hydrogenation of organic nitro compounds. It is advantageous that the bulk density of the Raney catalysts used is in the range from 0.3 g/ml to 3.0 g/ml.

If catalyst shaped articles which are too large are used, the educt to be hydrogenated possibly cannot come into contact with the catalyst to a sufficient extent. Too small a particle size of the catalysts means that a very high pressure loss, possibly too high, occurs in the continuous procedure. It is therefore preferable for the catalyst shaped articles used to have a diameter in the range from 0.05 to 20 mm.

So that the catalysts employed in the process according to the invention have on the one hand an adequate strength and on the other hand an optimized bulk density, it is preferable for the catalyst shaped articles used to have an activated shell thickness in the range from 0.05 to 7 mm, preferably 0.1 mm to 5 mm.

The catalyst shells can be impermeable or can have a porosity of 0% up to 80% and higher.

Catalysts in the form of hollow bodies which comprise one or more layers can be used in the process according to the invention. If the catalyst hollow bodies have several layers, the shaped articles can be dried between the individual coating steps during the preparation. This can be carried out in a fluidized bed at temperatures of 60 to 150° C. It is also possible for the catalyst shaped article will not to be dried between the first and second coating.

It is possible for the activated catalyst shaped articles employed in the process according to the invention to comprise at least one inorganic binder. The binder enables the catalyst bodies to have a higher strength. Preferably, powders of the metals which are also contained in the catalyst alloy as catalytically active constituents are added as binders during the preparation of the catalyst hollow bodies. However, it is also possible to add other binders, in particular other metals, as binders.

It is often also advantageous for the catalyst shaped articles employed in the process according to the invention to comprise no binder. If one of the cobalt catalysts is employed according to the invention for the preparation of substituted amines, these are preferably employed without a binder. Cobalt catalysts in the form of hollow bodies can have an adequate strength even without an added binder.

The catalyst alloy of the catalysts used according to the invention is preferably composed to the extent of 20-80 wt. % of one or more catalytically active metals and to the extent of 20-80 wt. % of one or more metals which can be leached out with alkalis, preferably aluminium. A rapidly or a slowly cooled alloy can be used as the catalyst alloy. Rapid cooling is understood as meaning, for example, cooling at a rate of 10 to 10⁵ K/s. Cooling media can be various gases or liquids, such as, for example, water. Slow cooling is understood as meaning methods with lower cooling rates.

Raney catalysts which are in the form of hollow bodies, extrudates, fibrous tablets or tablets and are doped with other metals can be used in the process according to the invention. The doping metals are often also called promoters. The doping of Raney catalysts is described, for example, in the documents U.S. Pat. No. 4,153,578, DE 21 01 856, DE 21 00 373 or DE 20 53 799. The cobalt catalyst used can be preferably doped with one or more elements from groups 3B to 7B, 8 and 1B of the periodic table, in particular chromium, manganese, iron, vanadium, tantalum, titanium, tungsten, molybdenum, rhenium and/or metals of the platinum group. It is also possible, but less preferred, for the cobalt catalyst used to be doped with one or more elements from groups 1A, 2A, 2B and/or 3A of the periodic table and/or germanium, tin, lead, antimony or bismuth. The content of promoters in the catalyst can preferably be 0-20 wt. %. The promoters can already be contained in the catalyst as an alloy constituent, or can be added only at a later point in time, in particular after the activation.

The Raney catalysts in the form of hollow bodies, extrudates, fibrous tablets or tablets are employed in the activated form during the process according to the invention. The metal which can be leached out and is present in the non-activated catalyst shaped articles can have been leached out with alkalis completely or only partly in the activated state.

The process according to the invention can be carried out with hydrogen as the hydrogenating gas or with gas mixtures which comprise hydrogen, for example a mixture of hydrogen and carbon monoxide, nitrogen and/or carbon dioxide. In order to avoid possible poisoning of the catalyst, it is preferable to carry out the process according to the invention with a gas or gas mixture comprising at least 95%, preferably at least 99% hydrogen.

The process allows the preparation of more or less pure individual substances and also the preparation of mixtures of various substituted amines.

It is preferable for the hydrogenation to be carried out in a fixed bed or suspension reactor in continuous operation. However, the invention also provides for carrying out the hydrogenation in the batch process. In the continuous procedure, the reactor can be operated in the liquid phase process or in the trickle bed process, the trickle bed process being preferred. Reactors and precise methods of carrying out the reaction are known.

The process according to the invention can be carried out with substituted aliphatic and aromatic nitro compounds. According to this invention, substituted compounds are understood as meaning those compounds which carry one or more radicals starting from unbranched n-alkanes, unbranched cycloalkanes, or mono- or polynuclear aromatics carrying exclusively hydrogen. These radicals can be, independently of one another, for example, alkyl, cycloalkyl, aryl, alkenyl, alkinyl F, Cl, Br, I, NO₂, NH₂, NHR, NR₂, OH, HS, S═C, R—CO—O, R—SO, R—SO2, CN, O═CR, HOOC, H₂NOC, ROOC or RO radicals, where R=alkyl, cycloalkyl, aryl, alkenyl, alkinyl, amino, alkylamino. The radicals R can optionally carry further substituents, such as, for example, alkyl, cycloalkyl, aryl, alkenyl, alkinyl F, Cl, Br, I, NO₂, NH₂, NHalkyl, NHaryl, Nalkyl₂, Naryl₂, OH, HS, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, cycloalkylO or arylO radicals.

The starting compound must be chosen such that the desired product can be obtained by hydrogenation of one or more nitro groups and optionally hydrogenation of other groupings accessible to hydrogenation.

Substituted aliphatic and aromatic amines can be prepared from the nitro compounds on which they are based by the process according to the invention. It is also possible to prepare, from nitro compounds, substituted amines in which at least one substituent is newly formed under the hydrogenation conditions according to the invention. Thus, for example, it is possible that during the preparation of the amine, a carbonyl groups contained in the nitro starting compounds is hydrogenated to a hydroxymethyl group at the same time, and a hydroxymethyl-substituted amine is thus formed.

The amines prepared according to the invention are substituted primary amines. It is possible that the products are amines with the general formula

R¹—A—NH₂

wherein A is a mono- or polynuclear aromatic, the free valencies of which are satisfied exclusively with hydrogen atoms, or A represents an open-chain, unbranched alkyl chain or a cyclic unbranched cycloalkyl group and R¹ is a substituent from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, RS, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO radicals or a heterocyclic radical. A can be an aromatic which carries exclusively carbon atoms or a heteroaromatic. The rings of the aromatics, regardless of whether they are mono- or polynuclear, can preferably be five- or six-membered. The positions of the radicals NH₂ and R¹ in the amine prepared according to the invention are not fixed. It is possible that they are vicinal, geminal, in the ortho-, para- or meta-position relative to one another or are arranged at a greater distance from one another. The radical R¹ can optionally also be substituted by one or more groups from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, alkyl-S, aryl-S, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, Sialkyl₃, cycloalkylO, arylO radicals or heterocyclic radical. It is furthermore possible for A also to contain one or more radicals in addition to R¹, it being possible for these radicals independently of one another and independently of the nature of the radical R¹ to originate from the series consisting of F, Cl, Br, I, NO₂, NH₂, NHalkyl, NHaryl, Nalkyl₂, Naryl₂, OH, HS, alkyl-S, aryl-S, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, cycloalkylO, arylO radicals or a heterocyclic radical.

It is preferable to obtain as the product substituted amines with the general formula R¹—A—NH₂, in which A is a benzene or naphthalene radical, in particular a benzene radical, and R¹ is a substituent from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, RS, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO radicals or a heterocyclic radical. In addition to the radical R¹, one or more other radicals can preferably also be bonded to the aromatic nucleus, in addition to hydrogen. Particularly preferred radicals are halogens, in particular chlorine, branched and unbranched alkyl radicals having 1-30 carbon atoms, for example methyl, ethyl, propyl, isopropyl, n-butyl, pentyl, hexyl, 2-ethylhexyl, 1,1-dimethylpropyl, decyl, dodecyl or octyl radicals, alkoxy radicals, such as, for example, methoxy or ethoxy radicals, R²—S groupings, where R²=C₁-C₁₈-alkyl, C₃-C₈-cycloalkyl or C7-C10-aralkyl, hydroxyl groups, acylated hydroxyl groups and free or acylated amino groups. The acylated groups can be derived, for example, from benzoic acid, 2-chlorobenzoic acid, 4-chlorobenzoic acid, p-hydroxybenzoic acid, p-methoxybenzoic acid or acetic acid. The radicals can be in the ortho-, meta- or para-position relative to the amino group.

According to the preferred embodiment of the process according to the invention, for example, o-toluidine, m-toluidine, p-toluidine, 4-methyl-1,3-phenylenediamine, from dinitrotoluene, 2-chloroaniline, 3-chloroaniline, 4-chloroaniline, 2,4-dichloroaniline, 2,3-dichloroaniline, the corresponding fluoro- and bromoanilines, 4-aminophenylacetic acid, 2-hydroxyaniline, 4-hydroxyaniline, 2-methoxyaniline, 4-methoxyaniline, various aminocresols, aminophenols, aminochlorophenols, aminobenzoic acids, aminohydroxybenzoic acids, aminochlorobenzoic acids, aminobenzophenones, substituted and unsubstituted aminophenylacetates, 2-amino-6-Chloroalkylsulfanes and 1-amino-5-Bromonaphthalene can be prepared.

It is also possible to prepare substituted aliphatic or cycloaliphatic amines by the process according to the invention. However, in many cases this possibility is limited by the accessibility of the starting compounds. Many aliphatic nitro compounds are relatively difficult to prepare or can be prepared only in relatively poor yields, so that it is often uneconomical to synthesize the appropriate amines by hydrogenation of the corresponding nitro compounds. Nevertheless, the process according to the invention can also be used for the preparation of substituted aliphatic amines with similarly good yields to those in the preparation of aromatic amines. However, more drastic conditions, for example higher temperatures and pressure, are often to be employed in the hydrogenation of aliphatic or cycloaliphatic substituted nitro compounds than in the case of aromatic nitro compounds, since aliphatically bonded nitro groups as a rule are less active with respect to hydrogenation.

Regardless of what type of substituted amines is to be prepared, according to the invention it is possible to prepare only one substituted amine in a reaction. However, it is also possible to prepare mixtures of various amines by the process according to the invention. These mixtures can be obtained, for example, by non-selective hydrogenation of starting substances which contain several nitro groups which can be hydrogenated or at least one nitro group and at least one other group which can be hydrogenated, or by hydrogenation of a mixture of substituted nitro compounds.

Depending on the starting compound, it is possible to carry out the process according to the invention in the liquid phase or in the gas phase. In general, however, it is preferable to carry out the process in the liquid phase. The process can be carried out in the liquid phase only if the compound to be hydrogenated is liquid or soluble in a solvent under the reaction conditions. In many cases it is preferable to carry out the reaction in the presence of a solvent. All the usual solvents can in principle be employed, as long as they do not interfere in the hydrogenation reaction. Examples of solvents which can be used are water, dioxane, acetone, methyl ethyl ketone, tetrahydrofuran, cyclohexane, methanol, ethanol, n-propanol, isopropanol, n-butanol, cyclohexanol, ethylene glycol, 1,4-butanediol, 1,6-hexanediol, ethylene glycol methyl ether, ethylene glycol ethyl ether, ethylene glycol dimethyl ether or triethylene glycol methyl ether. Mixtures of different solvents are also possible. The presence of one or more solvents can lead on the one hand to the operating parameters, such as pressure and temperature, lying in more moderate ranges than in the solvent-free procedure, or to the reaction being rendered possible in the first place. On the other hand, by skilful choice of the solvents, the selectivity of the hydrogenation reaction can be increased. Preferred solvents are alcohols, in particular methanol and isopropanol, and also toluene, tetrahydrofuran or cyclohexane. It may furthermore be advantageous to add bases, in particular alkali metal hydroxides and/or alkali metal alcoholates, to the solvent in order to increase the solubility of the substituted nitro compounds.

The process according to the invention is preferably carried out under an increased hydrogen pressure. The hydrogen pressures in the hydrogenation can conventionally be in a range between 1 and 300 bar, preferably between 2 and 100 bar.

The hydrogenation can be carried out in a temperature range between 0° C. and approximately 250° C., preferably between room temperature and 150° C., depending on the particular substituted nitro compound.

The amount of hydrogenation catalyst employed is usually non-critical. However, amounts of catalyst which are too low lead to long reaction times, while amounts of catalyst which are too high as a rule are uneconomical. According to the invention, for example, between 0.1 and 30 wt. % of catalyst moist weight, preferably 0.5-20 wt. %, particularly preferably 1-15 wt. %, based on the weight of substituted nitro compound to be hydrogenated, is employed,.

By varying the reaction conditions in the preparation of substituted amines by catalytic hydrogenation of substituted nitro compounds, it is possible also to hydrogenate other groups which can be hydrogenated and are present in the starting compound or not to hydrogenate the group which, in addition to the nitro group, is also accessible to a hydrogenation. It is thus possible on the one hand for the substituent or substituents present according to the invention in the substituted amine to be formed from another substituent by hydrogenation. On the other hand, however, it is also possible for substituted amines to be obtained by hydrogenation of nitro compounds which also contain as substituents other groups which can be hydrogenated. For example, from the same nitro compound substituted by a carboxylic acid ester group an amine substituted by the same ester group or an amine substituted by a hydroxymethyl group can be prepared by the process according to the invention, depending on the reaction parameters. An example of the preparation of a substituted aromatic amine by reduction of a substituted aromatic nitro compound is the synthesis of 2-aminobenzyl alcohol from 2-nitrobenzaldehyde.

The preparation of the substituted amines can particularly preferably be carried out continuously in the fixed bed process or semi-continuously. The process can be carried out here in the so-called trickle bed process or in the liquid phase process. The solution to be hydrogenated can be passed accordingly through the catalyst bed from the top or from the bottom. A co-current or a counter-current process can be employed here in a known manner. The catalyst loads can conventionally be in the range between 0.05 and 20 kg of substituted nitro compound per kg of catalyst and hour.

In the case of the continuous procedure, it is also possible to carry out the hydrogenation in two or more stages. For example, the hydrogenation can be carried out in a first stage at a temperature in the range between 20 and 60° C., and can be completed in a second stage at a temperature in the range from 50 to 100° C. The formation of by-products, for example, can be reduced in this manner.

However, the invention also provides for carrying out the hydrogenation in the suspension process or in the batch process in the manner in which the catalyst is arranged in a fixed form in a catalyst basket. Suitable reactors for the procedures mentioned are known from the prior art. In this case the amount of hydrogenation catalyst employed is usually non-critical. However, amounts of catalyst which are too low lead to long reaction times, while amounts of catalyst which are too high as a rule are uneconomical. According to the invention, for example, between 0.1 and 30 wt. %, preferably 0.5-20 wt. %, particularly preferably 1-15 wt. % of catalyst moist weight, based on the weight of substituted nitro compound to be hydrogenated, is employed.

If the substituted amines are prepared in the batch process by the process according to the invention, it is not absolutely necessary for the entire amount of the substituted nitro compound to be hydrogenated to be in solution. Some of the nitro compound can also be present as a solid in suspension, it being gradually dissolved and hydrogenated during the reaction. In such a case it may also be helpful to carry out the reaction with the addition of a phase transfer catalyst.

In particular cases, for example in the preparation of aromatic halogenoamines, it may be advantageous to add to the reaction mixture a sulfur compound, for example thiourea. The selectivity of the amine preparation can be improved by addition of the sulfur compounds in that hydrogenolytic splitting off of the halogens is reduced.

One embodiment of the process according to the invention for the preparation of substituted amines by catalytic hydrogenation of substituted organic nitro compounds with the aid of Raney catalysts in the form of hollow bodies has the following advantages:

The Raney catalyst in the form of hollow bodies used according to the invention has a significantly lower bulk density than the Raney catalysts used hitherto. As a result, considerably less catalyst material is required than in the processes known hitherto.

In spite of the significantly smaller amount of catalyst material, the preparation of substituted amines can be carried out with high conversion rates, very good yields and very good space/time yields.

Another embodiment of the process according to the invention for the preparation of substituted amines by catalytic hydrogenation of substituted organic nitro compounds with the aid of Raney catalysts in the form of extrudates, fibrous tablets or tablets has the following advantages:

The Raney catalyst in the form of extrudates, fibrous tablets or tablets which is used according to the invention has a significantly higher heat exchange capacity than known supported catalysts. As a result, these catalysts will be suitable for highly exothermic reactions.

The catalyst employed in the process according to the invention has a very good strength. This results in a very good hydrogenation activity which lasts a relatively long time, so that long running times without interruptions are achieved in continuous operation.

Because of its state, the catalyst is easy to separate off from the reaction medium.

### USE EXAMPLE 1

The catalytic activities of the catalyst of examples 1 to 9 during hydrogenation of dinitrotoluene (DNT) were compared. For this purpose, 40 ml catalyst (from 31 to 77 grams of the corresponding catalysts) were introduced into a tube reactor and tested in a trickle phase. The temperature range of the reaction was 80 to 120° C., the concentration of the DNT in methanol was 0.5 to 6.0 wt. % and the pressure of the reaction was always 60 bar. The throughput of hydrogen was 75 to 150 l/h and the throughput of DNT was 0.01 to 2.5 g DNT/h·ml of catalyst. The product mixture was analysed by GC.

### EXAMPLE 1

A coating solution was prepared by suspending 1730 grams of 48.5% Ni, 50.1% Al, 0.9% Cr and 0.5% Fe alloy powder (this alloy was melted in an induction furnace, poured into a crucible, cooled in air and comminuted) and 130 grams of pure nickel powder (99% Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 48.5% Ni, 50.5% Al, 0.9% Cr and 0.5% Fe alloy powder (this alloy was melted in an induction furnace, poured into a crucible, cooled in air and comminuted), 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al/Cr/Fe, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al/Cr/Fe hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. 40 ml (31.09 grams) of this catalyst were tested in accordance with use example 1 and the results of this experiment are shown in table 1.

TABLE 1
The test results for example 1.

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 100.1 | 75 | 0.035 | 2 | 100 | 90.2 | 0.251 | 0.195 |
| 100.3 | 90 | 0.036 | 2 | 100 | 90.1 | 0.255 | 0.198 |
| 100.1 | 90 | 0.036 | 2 | 99.7 | 91.3 | 0.252 | 0.196 |
| 90.5 | 90 | 0.036 | 2 | 99.7 | 95.0 | 0.252 | 0.196 |
| 80.7 | 90 | 0.036 | 2 | 99.1 | 96.4 | 0.250 | 0.194 |
| 80.5 | 90 | 0.036 | 4 | 100 | 96.8 | 0.256 | 0.199 |
| 81.1 | 90 | 0.071 | 4 | 97.7 | 94.3 | 0.487 | 0.379 |

^{A}. litres/hour
^{B}. grams of DNT/(h · ml catalyst)
^{C}. % DNT in methanol
^{D}. % selectivity
^{E}. hydrogenated mmol of DNT/(h · g catalyst)
^{F}. hydrogenated mmol of DNT/(h · ml catalyst)

### EXAMPLE 2

A coating solution was prepared by suspending 1730 grams of 53% Ni and 47% Al alloy powder (this alloy was melted in an induction furnace, poured into a crucible, cooled in air and comminuted) and 130 grams of pure nickel powder (99% Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 53% Ni and 47% Al alloy powder (this alloy was melted in an induction furnace, poured into a crucible, cooled in air and comminuted), 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. 40 ml (39.09 grams) of this catalyst were tested in accordance with use example 1 and the results of this experiment are shown in table 2A.

TABLE 2A
The test results for example 2

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80 | 90 | 0.036 | 2 | 100 | 92.3 | 0.201 | 0.196 |
| 81 | 90 | 0.054 | 2 | 100 | 94.2 | 0.302 | 0.295 |
| 81 | 90 | 0.070 | 2 | 99.0 | 93.1 | 0.392 | 0.383 |
| 81 | 90 | 0.143 | 4 | 99.0 | 94.0 | 0.797 | 0.779 |
| 101 | 90 | 0.148 | 4 | 100 | 89.1 | 0.831 | 0.812 |

A. liters/hour

B. grams of DNT/(h·ml catalyst)

C. %DNT in methanol

D. %selectivity

E. hydrogenated mmol of DNT/(h·g catalyst)

F. hydrogenated mmol of DNT/(h·ml catalyst)

After the first series of experiments the catalyst was deactivated with 100 ml of a 2.8 wt. % NaNO₃ for 5 minutes under a gentle stream of H₂ under normal pressure at 80° C. The catalyst was then washed and the reaction according to use example 1 was started again. The results of this experiment with the deactivated catalyst are shown in table 2B.

TABLE 2B
The test results for the deactivated catalyst of example 2

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80 | 90 | 0.036 | 2 | 100 | 96.7 | 0.202 | 0.197 |
| 81.9 | 90 | 0.069 | 2 | 99.1 | 94.3 | 0.381 | 0.372 |
| 81.8 | 90 | 0.072 | 2 | 98.2 | 93.4 | 0.397 | 0.388 |
| 82.7 | 90 | 0.143 | 4 | 96.6 | 90.9 | 0.778 | 0.760 |
| 101.9 | 90 | 0.153 | 4 | 99.3 | 91.8 | 0.852 | 0.833 |

^{A}. litres/hour
^{B}. grams of DNT/(h · ml catalyst)
^{C}. % DNT in methanol
^{D}. % selectivity
^{B}. hydrogenated mmol of DNT/(h · g catalyst)
^{F}. hydrogenated mmol of DNT/(h · ml catalyst)

### EXAMPLE 3

A coating solution was prepared by suspending 1730 grams of 53% Ni and 47% Al alloy powder (this alloy was melted in an induction furnace, poured into a crucible, cooled in air and comminuted) and 130 grams of pure nickel powder (99% Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 53% Ni and 47% Al alloy powder (this alloy was melted in an induction furnace, poured into a crucible, cooled in air and comminuted), 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. This catalyst was doped with a vanadium chloride solution in the presence of NaOH. The V content of the catalyst at the end was 0.3%. 40 ml (34.04 grams) of this catalyst were tested in accordance with use example 1 and the results of this experiment are shown in table 3.

TABLE 3
The test results for example 3

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80 | 90 | 0.072 | 4 | 100 | 95.6 | 0.467 | 0.397 |
| 81 | 90 | 0.108 | 4 | 99.7 | 96.6 | 0.697 | 0.593 |
| 81 | 90 | 0.144 | 4 | 98.9 | 96.5 | 0.920 | 0.783 |
| 83 | 90 | 0.238 | 4 | 95.3 | 96.1 | 1.464 | 1.246 |
| 80 | 90 | 0.048 | 4 | 100 | 95.6 | 0.310 | 0.264 |

^{A}. litres/hour
^{B}. grams of DNT/(h · ml catalyst)
^{C}. % DNT in methanol
^{D}. % selectivity
^{E}. hydrogenated mmol of DNT/(h · g catalyst)
^{F}. hydrogenated mmol of DNT/(h · ml catalyst)

### EXAMPLE 4

A free-flowing, pelletable catalyst mixture was prepared in accordance with the instructions in EP 0 648 534 A1 for a catalyst of 1000 grams of 53% Ni and 47% Al alloy powder (this alloy was melted in an induction furnace, poured into a crucible, cooled in air and comminuted), 150 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tablets were activated for 2 hours at 80° C. in 20% sodium hydroxide solution. 40 ml (76.21 grams) of this catalyst were tested in accordance with use example 1 and the results of this experiment are shown in table 4.

TABLE 4
The test results for example 4

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 82 | 90 | 0.073 | 4 | 98.7 | 94.5 | 0.208 | 0.396 |
| 82 | 90 | 0.108 | 4 | 94.9 | 92.3 | 0.296 | 0.564 |
| 82 | 90 | 0.143 | 4 | 90.2 | 89.7 | 0.373 | 0.711 |
| 84 | 90 | 0.239 | 4 | 83.0 | 87.2 | 0.572 | 1.010 |
| 81 | 90 | 0.048 | 4 | 99.0 | 95.5 | 0.138 | 0.263 |

^{A}litres/hour
^{B}grams of DNT/(h · ml catalyst)
^{C}% DNT in methanol
^{D}% selectivity
^{E}hydrogenated mmol of DNT/(h · g catalyst)
^{F}hydrogenated mmol of DNT/(h · ml catalyst)

### EXAMPLE 5

A coating solution was prepared by suspending 1730 grams of 53% Ni and 47% Al alloy powder (this alloy was melted in an induction furnace, poured into a crucible, cooled in air and comminuted) and 130 grams of pure nickel powder (99% Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 53% Ni and 47% Al alloy powder (this alloy was melted in an induction furnace, poured into a crucible, cooled in air and comminuted), 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. This catalyst was doped with a palladium nitrate solution adjusted to pH 6 with Na₂CO₃. The Pd content of the catalyst at the end was 0.3%. 40 ml (34.78 grams) of this catalyst were tested in accordance with use example 1 and the results of this experiment are shown in table 5.

TABLE 5
The test results for example 5.

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80 | 90 | 0.072 | 4 | 100 | 93.9 | 0.455 | 0.396 |
| 80 | 90 | 0.107 | 4 | 100 | 94.8 | 0.673 | 0.585 |
| 80 | 90 | 0.146 | 4 | 100 | 95.8 | 0.923 | 0.803 |
| 81 | 90 | 0.233 | 4 | 100 | 96.3 | 1.474 | 1.282 |
| 80 | 90 | 0.048 | 4 | 100 | 94.3 | 0.306 | 0.266 |
| 81 | 90 | 0.320 | 4 | 100 | 94.1 | 2.022 | 1.758 |
| 81 | 90 | 0.353 | 4 | 100 | 96.6 | 2.229 | 1.938 |
| 83 | 90 | 0.464 | 4 | 100 | 96.9 | 2.929 | 2.547 |
| 83 | 90 | 0.443 | 4 | 100 | 97.3 | 2.795 | 2.430 |
| 87.5 | 90 | 0.688 | 4 | 98.3 | 96.6 | 4.267 | 3.710 |
| 105.7 | 90 | 0.699 | 4 | 100 | 93.6 | 4.416 | 3.840 |

^{A}. litres/hour
^{B}. grams of DNT/(h · ml catalyst)
^{C}. % DNT in methanol
^{D}. % selectivity
^{E}. hydrogenated mmol of DNT/(h · g catalyst)
^{F}. hydrogenated mmol of DNT/(h · ml catalyst)

### EXAMPLE 6

A free-flowing, pelletable catalyst mixture was prepared in accordance with the instructions in EP 0 648 534 A1 for a catalyst of 1000 grams of 40% Ni, 58.5% Al, 1.0% Cr and 0.5% Fe alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tablets were activated for 2 hours at 80° C. in 20% sodium hydroxide solution. 40 ml (50.44 grams) of this catalyst were tested in accordance with use example 1 and the results of this experiment are shown in table 6.

TABLE 6
The test results for example 6.

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80.5 | 90 | 0.072 | 4 | 100 | 96.4 | 0.313 | 0.395 |
| 80.7 | 90 | 0.144 | 4 | 100 | 97.9 | 0.628 | 0.792 |
| 81.8 | 90 | 0.239 | 4 | 100 | 97.9 | 1.043 | 1.315 |
| 83.2 | 90 | 0.312 | 4 | 100 | 97.9 | 1.360 | 1.715 |
| 86.0 | 90 | 0.462 | 4 | 100 | 97.9 | 2.011 | 2.536 |
| 91.5 | 90 | 0.703 | 4 | 100 | 97.9 | 3.059 | 3.856 |
| 97.5 | 90 | 1.063 | 4 | 100 | 97.2 | 4.628 | 5.836 |
| 90.0 | 90 | 1.060 | 6 | 100 | 98.0 | 4.614 | 5.818 |
| 93.8 | 90 | 1.383 | 6 | 100 | 97.1 | 6.022 | 7.594 |

^{A}litres/hour
^{B}grams of DNT/(h · ml catalyst)
^{C}% DNT in methanol
^{D}% selectivity
^{E}hydrogenated mmol of DNT/(h · g catalyst)
^{F}hydrogenated mmol of DNT/(h · ml catalyst)

### EXAMPLE 7

A free-flowing, pelletable catalyst mixture was prepared in accordance with the instructions in EP 0 648 534 A1 for a catalyst of 1000 grams of 50% Ni and 50% Al alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tablets were activated for 2 hours at 80° C. in 20% sodium hydroxide solution. 40 ml (69.7 grams) of this catalyst were tested in accordance with use example 1 and the results of this experiment are shown in table 7.

TABLE 7
The test results for example 7

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80.3 | 90 | 0.072 | 4 | 100 | 97.1 | 0.226 | 0.394 |
| 80.4 | 90 | 0.144 | 4 | 100 | 97.6 | 0.453 | 0.789 |
| 80.8 | 90 | 0.240 | 4 | 100 | 97.9 | 0.756 | 1.317 |
| 81.6 | 90 | 0.313 | 4 | 100 | 97.5 | 0.985 | 1.716 |
| 83.4 | 90 | 0.463 | 4 | 100 | 96.7 | 1.459 | 2.542 |
| 85.8 | 90 | 0.718 | 4 | 98.6 | 94.6 | 2.230 | 3.886 |
| 89.1 | 150 | 0.909 | 4 | 95.7 | 91.9 | 2.738 | 4.771 |
| 89.5 | 150 | 1.155 | 6 | 98.4 | 93.7 | 3.581 | 6.240 |
| 99.6 | 150 | 1.455 | 6 | 96.4 | 91.8 | 4.422 | 7.705 |

^{A}litres/hour
^{B}grams of DNT/(h · ml catalyst)
^{C}% DNT in methanol
^{D}% selectivity
^{E}hydrogenated mmol of DNT/(h · g catalyst)
^{F}hydrogenated mmol of DNT/(h · ml catalyst)

### EXAMPLE 8

A free-flowing, pelletable catalyst mixture was prepared in accordance with the instructions in EP 0 648 534 A1 for a catalyst of 1000 grams of 40% Ni, 58.5% Al, 1.0% Cr and 0.5% Fe alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tablets were activated for 2 hours at 80° C. in 20% sodium hydroxide solution. This catalyst was doped with a palladium nitrate solution adjusted to pH 6 with Na₂CO₃. The Pd content of the catalyst at the end was 0.2%. 40 ml (58.87 grams) of this catalyst were tested in accordance with use example 1 and the results of this experiment are shown in table 8.

TABLE 8
The test results for example 8

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 80.2 | 90 | 0.112 | 6 | 100 | 96.5 | 0.418 | 0.615 |
| 80.6 | 90 | 0.219 | 6 | 100 | 97.0 | 0.816 | 1.201 |
| 81.2 | 90 | 0.363 | 6 | 100 | 97.2 | 1.353 | 1.991 |
| 85.3 | 90 | 0.717 | 6 | 100 | 97.2 | 2.676 | 3.938 |
| 91.5 | 90 | 1.060 | 6 | 100 | 97.4 | 3.954 | 5.819 |
| 94.7 | 150 | 1.429 | 6 | 100 | 96.6 | 5.330 | 7.844 |
| 109.0 | 150 | 2.155 | 6 | 99.9 | 94.6 | 8.006 | 11.784 |
| 114.0 | 150 | 2.489 | 6 | 99.5 | 95.0 | 9.233 | 13.589 |
| 129.8 | 150 | 2.487 | 6 | 99.8 | 92.3 | 9.258 | 13.626 |

^{A}litres/hour
^{B}grams of DNT/(h · ml catalyst)
^{C}% DNT in methanol
^{D}% selectivity
^{E}hydrogenated mmol of DNT/(h · g catalyst)
^{F}hydrogenated mmol of DNT/(h · ml catalyst)

### EXAMPLE 9

A coating solution was prepared by suspending 1730 grams of 40% Ni, 58.5% Al, 1.0% Cr and 0.5% Fe alloy powder (this alloy was melted in an induction furnace and sprayed with water) and 130 grams of pure nickel powder (99% Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 40% Ni, 58.5% Al, 1.0% Cr and 0.5% Fe alloy powder (this alloy was melted in an induction furnace and sprayed with water), 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al/Cr/Fe, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al/Cr/Fe hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. This catalyst was doped with a palladium nitrate solution adjusted to pH 6 with Na₂CO₃. The Pd content of the catalyst at the end was 0.3%. 40 ml (32.68 grams) of this catalyst were tested in accordance with use example 9 and the results of this experiment are shown in table 9.

TABLE 9
The test results for example 9

| | H₂ | DNT | | % Con- | | Activity | Activity |
|---|---|---|---|---|---|---|---|
| ° C. | throughput^{A} | throughput^{B} | % DNT^{C} | version | % Sel^{D} | per gram^{E} | per ml^{F} |
| 81.3 | 90 | 0.110 | 6 | 100 | 97.0 | 0.738 | 0.603 |
| 83.0 | 90 | 0.218 | 6 | 96.3 | 95.5 | 1.414 | 1.155 |
| 86.0 | 90 | 0.366 | 6 | 92.5 | 89.0 | 2.273 | 1.857 |
| 90.5 | 90 | 0.705 | 6 | 89.8 | 76.9 | 4.258 | 3.479 |
| 110.8 | 90 | 0.727 | 6 | 94.4 | 92.7 | 4.613 | 3.769 |
| 118.0 | 90 | 1.326 | 6 | 89.3 | 87.0 | 7.958 | 6.502 |

^{A}. litres/hour
^{B}. grams of DNT/(h · ml catalyst)
^{C}. % DNT in methanol
^{D}. % selectivity
^{E}. hydrogenated mmol of DNT/(h · g catalyst)
^{F}. hydrogenated mmol of DNT/(h · ml catalyst)

1. Process for the preparation of substituted amines by catalytic hydrogenation of substituted organic nitro compounds with hydrogen or hydrogen-containing gas mixtures in the presence of a shaped Raney catalyst as the hydrogenation catalyst, characterized in that the Raney catalyst is in the form of hollow bodies, extrudates, granules, fibrous tablets or tablets, and in that the catalyst is prepared from rapidly and/or slowly cooled alloys.

2. Process according to claim 1, characterized in that the Raney catalysts in the form of hollow bodies, extrudates, granules, fibrous tablets or tablets comprise nickel, cobalt, copper, iron, platinum, palladium, ruthenium and/or mixtures of these metals as catalytically active constituents.

3. Process according to claim 1 or 2, characterized in that the Raney catalyst is in the form of hollow spheres or shell-activated tablets.

4. Process according to claim 1 or 3, characterized in that the bulk density of the Raney catalysts used is in the range from 0.3 g/ml to 3.0 g/ml.

5. Process according to claim 1 or 4, characterized in that the catalyst shaped articles used have a diameter in the range from 0.05 to 20 mm.

6. Process according to one or more of claims 1 to 5, characterized in that the catalyst shaped articles used have an activated shell thickness in the range from 0.05 to 7 mm, preferably 0.1 mm to 5 mm.

7. Process according to one or more of claims 1 to 5, characterized in that the catalyst shaped articles used in the process comprise at least one inorganic binder.

8. Process according to one or more of claims 1 to 6, characterized in that the catalyst shaped articles used in the process comprise no binder.

9. Process according to one or more of claims 1 to 8, characterized in that the cobalt catalyst used is doped with one or more elements from groups 3B to 7B, 8 and 1B of the periodic table, in particular chromium, manganese, iron, vanadium, tantalum, titanium, tungsten, molybdenum, rhenium, palladium and/or metals of the platinum group.

10. Process according to one or more of claims 1 to 9, characterized in that the cobalt catalyst used is doped with one or more elements from groups 1A, 2A, 2B and/or 3A of the periodic table and/or germanium, tin, lead, antimony or bismuth.

11. Process according to one or more of claims 1 to 8, characterized in that the nickel catalyst used is doped with one or more elements from groups 3B to 7B, 8 and 1B of the periodic table, in particular chromium, manganese, iron, vanadium, tantalum, titanium, tungsten, molybdenum, rhenium, palladium and/or metals of the platinum group.

12. Process according to one or more of claims 1 to 9, characterized in that the nickel catalyst used is doped with one or more elements from groups 1A, 2A, 2B and/or 3A of the periodic table and/or germanium, tin, lead, antimony or bismuth.

13. Process according to one or more of claims 1 to 12, characterized in that the hydrogenation is carried out in a fixed bed or suspension reactor in continuous operation.

14. Process according to one or more of claims 1 to 12, characterized in that the hydrogenation is carried out in the batch process.

15. Process according to one or more of claims 1 to 14, characterized in that the products are amines with the general formula R¹—A—NH₂, wherein A is a mono- or polynuclear aromatic, the free valencies of which are satisfied exclusively with hydrogen atoms, or A represents an open-chain, unbranched alkyl chain or a cyclic unbranched cycloalkyl group and R¹ is a substituent from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, RS, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO radicals or a heterocyclic radical.

16. Process according to one or more of claims 1 to 14, characterized in that substituted amines with the general formula R¹—A—NH₂, in which A is a benzene or naphthalene radical, in particular a benzene radical, and R¹ is a substituent from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, RS, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO radicals or a heterocyclic radical, are obtained as the product.

17. Process according to claim 16, characterized in that in addition to the radical R¹, one or more other radicals can be bonded to the aromatic nucleus, in addition to hydrogen.

18. Process according to one or more of claims 1 to 14, for the hydrogenation of 2,4-/2,6-dinitrotoluene mixtures.

19. Process according to one or more of claims 1 to 14 for the hydrogenation of 2,4-/2,6-dinitrotoluene mixtures, characterized in that the hydrogenation uses methanol or toluenediamine as the solvent, and under the conditions of 1 to 150 bar, and 20 to 200° C. [sic]

20. Process according to one or more of claims 1 to 14 for the hydrogenation of 2,4-/2,6-dinitrotoluene mixtures, characterized in that the hydrogenation uses methanol or toluenediamine as the solvent, and with the conditions of 5 to 80 bar, and 60 to 140° C.

21. Process with a Raney catalyst in the form of hollow bodies, extrudates, granules, fibrous tablets or tablets which comprises nickel, cobalt, copper, iron, platinum, palladium, ruthenium and/or mixtures of these metals as catalytically active constituents, for the hydrogenation of 2,4-/2,6-dinitrotoluene mixtures, characterized in that the hydrogenation uses methanol or toluenediamine as the solvent, and under the conditions of 1 to 150 bar, and 20 to 200° C., the catalyst preferably being prepared [sic] rapidly and/or slowly cooled alloys.

22. Process with a Raney catalyst in the form of hollow bodies, extrudates, granules, fibrous tablets or tablets which comprises nickel, cobalt, copper, iron, platinum, palladium, ruthenium and/or mixtures of these metals as catalytically active constituents, for the hydrogenation of 2,4-/2,6-dinitrotoluene mixtures, characterized in that the hydrogenation uses methanol or toluenediamine as the solvent, and under the conditions of 5 to 80 bar, and 60 to 140° C., the catalyst preferably being prepared from rapidly and/or slowly cooled alloys.

23. Process with a Raney catalyst in the form of hollow bodies, extrudates, granules, fibrous tablets or tablets which comprises nickel, cobalt, copper, iron, platinum, palladium, ruthenium and/or mixtures of these metals as catalytically active constituents, for the hydrogenation of 2,4-/2,6-dinitrotoluene mixtures, characterized in that the hydrogenation uses methanol or toluenediamine as the solvent, and under the conditions of 1 to 150 bar, and 20 to 200° C., the catalyst preferably being prepared from rapidly and/or slowly cooled alloys and the Raney catalyst used being doped with one or more elements from groups 3B to 7B, 8 and 1B of the periodic table, in particular chromium, manganese, iron, vanadium, tantalum, titanium, tungsten, molybdenum, rhenium, palladium and/or metals of the platinum group.

24. Process with a Raney catalyst in the form of hollow bodies, extrudates, granules, fibrous tablets or tablets which comprises nickel, cobalt, copper, iron, platinum, palladium, ruthenium and/or mixtures of these metals as catalytically active constituents, for the hydrogenation of 2,4-/2,6-dinitrotoluene mixtures, characterized in that the hydrogenation uses methanol or toluenediamine as the solvent, and with the conditions of 5 to 80 bar, and 60 to 140° C., the catalyst preferably being prepared from rapidly and/or slowly cooled alloys and the Raney catalyst used being doped with one or more elements from groups 3B to 7B, 8 and 1B of the periodic table, in particular chromium, manganese, iron, vanadium, tantalum, titanium, tungsten, molybdenum, rhenium, palladium and/or metals of the platinum group.

25. Process with a Raney catalyst in the form of hollow bodies, extrudates, granules, fibrous tablets or tablets . . . which comprises nickel, cobalt, copper, iron, platinum, palladium, ruthenium and/or mixtures of these metals as catalytically active constituents, for the hydrogenation of 2,4-/2,6-dinitrotoluene mixtures, characterized in that the hydrogenation uses methanol or toluenediamine as the solvent, and under the conditions of 1 to 150 bar, and 20 to 200° C., the catalyst preferably being prepared from rapidly and/or slowly cooled alloys and the Raney catalyst used being doped with one or more elements from groups 1A, 2A, 2B and/or 3A of the periodic table and/or germanium, tin, lead, antimony or bismuth.

26. Process with a Raney catalyst in the form of hollow bodies, extrudates, granules, fibrous tablets or tablets [sic] comprises nickel, cobalt, copper, iron, platinum, palladium, ruthenium and/or mixtures of these metals as catalytically active constituents, for the hydrogenation of 2,4-/2,6-dinitrotoluene mixtures, characterized in that the hydrogenation uses methanol or toluenediamine as the solvent, and under the conditions of 5 to 80 bar, and 60 to 140° C., the catalyst preferably being prepared from rapidly and/or slowly cooled alloys and the Raney catalyst used being doped with one or more elements from groups 1A, 2A, 2B and/or 3A of the periodic table and/or germanium, tin, lead, antimony or bismuth.

27. Process with a nickel Raney catalyst, in the form of hollow bodies, extrudates, granules, fibrous tablets or tablets, for the hydrogenation of 2,4-/2,6-dinitrotoluene mixtures, characterized in that the hydrogenation uses methanol or toluenediamine as the solvent, and under the conditions of 1 to 150 bar, and 20 to 200° C. [sic]

28. Process with a nickel Raney catalyst, in the form of hollow bodies, extrudates, granules, fibrous tablets or tablets, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

29. Process with a nickel Raney catalyst which is in the form of hollow bodies, extrudates, granules, fibrous tablets or tablets and is doped with Fe,Cr, Pd or V, for the hydrogenation of 2,4-/2,6-dinitrotoluene mixtures, characterized in that the hydrogenation uses methanol or toluenediamine as the solvent, and under the conditions of 1 to 150 bar, and 20 to 200° C. [sic]

30. Process with a nickel Raney catalyst which is in the form of hollow bodies, extrudates, granules, fibrous tablets or tablets and is doped with Fe,Cr, Pd or V, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

31. Process with a nickel Raney catalyst which is in the form of hollow bodies and is doped with Fe,Cr, Pd or V, for the hydrogenation of 2,4-/2,6-dinitrotoluene mixtures, characterized in that the hydrogenation uses methanol or toluenediamine as the solvent, and under the conditions of 1 to 150 bar, and 20 to 200° C. [sic]

32. Process with a nickel Raney catalyst which is in the form of hollow bodies and is doped with Fe,Cr, Pd or V, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

33. Process with a shell-activated nickel Raney catalyst which is in the form of tablets and is doped with Fe,Cr, Pd or V, for the hydrogenation of 2,4-/2,6-dinitrotoluene mixtures, characterized in that the hydrogenation uses methanol or toluenediamine as the solvent, and under the conditions of 1 to 150 bar, and 20 to 200° C.

34. Process with a shell-activated nickel Raney catalyst which is in the form of tablets and is doped with Fe,Cr, Pd or V, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

35. Process with a nickel Raney catalyst which is in the form of hollow bodies, is prepared from water-sprayed alloy and is doped with Fe,Cr, Pd or V, for the hydrogenation of 2,4-/2,6-dinitrotoluene mixtures, characterized in that the hydrogenation uses methanol or toluenediamine as the solvent, and under the conditions of 1 to 150 bar, and 20 to 200° C. [sic]

36. Process with a nickel Raney catalyst which is in the form of hollow bodies, is prepared from water-sprayed alloy and is doped with Fe,Cr, Pd or V, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

37. Process with a shell-activated nickel Raney catalyst which is in the form of tablets, is prepared from water-sprayed alloy and is doped with Fe,Cr, Pd or V, for the hydrogenation of 2,4-/2,6-dinitrotoluene mixtures, characterized in that the hydrogenation uses methanol or toluenediamine as the solvent, and under the conditions of 1 to 150 bar, and 20 to 200° C.

38. Process with a shell-activated nickel Raney catalyst which is in the form of tablets, is prepared from water-sprayed alloy and is doped with Fe,Cr, Pd or V, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

39. Process with a shell-activated nickel Raney catalyst which is in the form of tablets and is prepared from water-sprayed alloy, for the hydrogenation of 2,4-/2,6-dinitrotoluene mixtures, characterized in that the hydrogenation uses methanol or toluenediamine as the solvent, and under the conditions of 1 to 150 bar, and 20 to 200° C.

40. Process with a shell-activated nickel Raney catalyst which is in the form of tablets and is prepared from water-sprayed alloy, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.

41. Process with a nickel Raney catalyst which is in the form of hollow bodies and is prepared from water-sprayed alloy, for the hydrogenation of 2,4-/2,6-dinitrotoluene mixtures, characterized in that the hydrogenation uses methanol or toluenediamine as the solvent, and under the conditions of 1 to 150 bar, and 20 to 200° C.

42. Process with a nickel Raney catalyst which is in the form of hollow bodies and is prepared from water-sprayed alloy, for the hydrogenation of 2,4-/2,6-dinitrotoluene mixtures, characterized in that the hydrogenation uses methanol or toluenediamine as the solvent, and under the conditions of 5 to 80 bar, and 60 to 140° C.
